(19) United States
(12) Patent Application Publication
(10) Pub. No.: US 2006/0054171 A1
(43) Pub. Date: Mar. 16, 2006

(54) Method and apparatus of approaching a joint

(76) Inventor: Bruce Dall, Kalamazoo, MI (US)

Correspondence Address: POLSTER, LIEDER, WOODRUFF & LUCCHESI, 12412 POWERSCOURT DRIVE SUITE 200, ST. LOUIS, MO, 63131-3615, US

(21) Appl. No.: 10/941,464

(22) Filed: Sep. 15, 2004

(51) Int. Cl. A61B 19/00

(52) U.S. Cl. 128/898

(57) ABSTRACT

A method and apparatus for approaching a joint. The method of approaching a joint which connects a first bone and a second bone comprises cutting an incision to the joint and inserting a guide through the incision and into the joint. Next cartilage and bone portions are removed surrounding the guide to form a passage into the joint. A system for approaching a sacroiliac joint which connects an iliac bone and a sacrum bone comprises a central processing unit and a scanner in communication with the central processing unit. The scanner is adapted to analyze the sacroiliac joint for an insertion point within the sacroiliac joint. The system further comprises a guide in communication with the central processing unit wherein the guide is adapted to be inserted substantially parallel to the iliac bone and the sacrum bone and into the insertion point. A remover is adapted to form a passage around the guide and into the insertion point.

### BACKGROUND

The present disclosure relates to a method and apparatus for approaching a joint area. In particular, the present disclosure relates to a method and apparatus for approaching the sacroiliac joint in a minimally invasive manner.

A joint is the point of articulation between two or more bones, especially such as a connection of bones that allows motion. Ligaments are a sheet or band of tough fibrous tissue connecting bones at the joint. Most joints are designed for motion where the ligaments stretch and recoil between the moving bones. Due to the joint movements, ligaments may become injured, disrupted or torn leading to pain and inflammation of the joints. Eventually, the movements can lead to wear and tear of the joint and can lead to pain from degenerative arthritis.

A typical form of treatment for injured joints comprises invasive surgery which consists of inserting hardware such as screws across the joint or next to the joint. During surgery, the joint is opened via incisions through the skin and muscles so that the surgeon can see the joint and exposed bone surfaces. For this surgery treatment, the bones are held together until the bones fuse together since the body treats the exposed bone surfaces as a fracture. To hold the bones together, the surgeon will insert several metal screws across the joint, wherein bone graft may be placed around the screw to assist in fusing the joint.

Surgery, however, results in a variety of risk complications, especially any surgery performed near the spine or spinal cord. These surgery complications include unexpected blood loss, nerve damage, muscle damage and infections. Additionally, the surgery results in a duration of recovery and post operative care. In joint surgery, further complications arise due to the screw hardware. The implanted screws may move after surgery and begin to “back out.” Additionally, the screws may break if the fusion does not become solid. Accordingly, hardware removal and reoperation are often necessary for current joint surgeries.

### SUMMARY

The present disclosure relates to a method and apparatus for approaching a joint in a minimally invasive manner. In an embodiment, the present disclosure relates to a method of approaching a joint which may connect adjacent bones. The method comprises cutting an incision through skin and ligaments in a region overlaying the joint. Next, a guide may be inserted through one of the bones and into the joint. Cartilage and bone portions connected to the cartilage surrounding the guide are then removed to form a passage to the joint to allow treatment of the joint.

In an embodiment, the present disclosure relates to a system for approaching the joint which may connect adjacent bones. The system comprises a central processing unit and a scanner in communication with the central processing unit. The scanner is adapted to analyze the joint for an insertion point within the joint. The system further comprises a guide in communication with the central processing unit wherein the guide may be inserted parallel or substantially parallel to the bones and into the insertion point. A remover is adapted to form a passage around the guide and into the insertion point wherein the passage is configured for any suitable non-fusion or fusion device to treat the joint.

### BRIEF DESCRIPTION OF THE DRAWINGS

The detailed description particularly refers to the accompanying figures in which:

FIG. 1 is a front elevational view of a skeletal structure of the pelvic region;

FIG. 2 is a partial front elevational view of a sacroiliac joint of FIG. 1;

FIG. 3 is a planar cross sectional view of the sacroiliac joint of FIG. 2;

FIG. 4 is a flowchart illustrating steps of approaching the sacroiliac joint in accordance with an embodiment of the present disclosure;

FIG. 5 is a side sectional view of the pelvis of FIG. 2 showing a guide procedure involved in approaching the sacroiliac joint of FIG. 2;

FIG. 6 is a cross sectional view of FIG. 5 showing a cannulated removing procedure;

FIG. 7 is a side sectional view of a pelvis showing a removing procedure involved in approaching the sacroiliac joint of FIG. 2;

FIG. 8 is a cross sectional view of FIG. 7 showing the removing procedure;

FIG. 9 is a cross sectional view of FIG. 6 showing a probe procedure; and

FIG. 10 is a schematic view of a system for approaching the sacroiliac joint in accordance with the present disclosure.

### DESCRIPTION OF THE DISCLOSURE

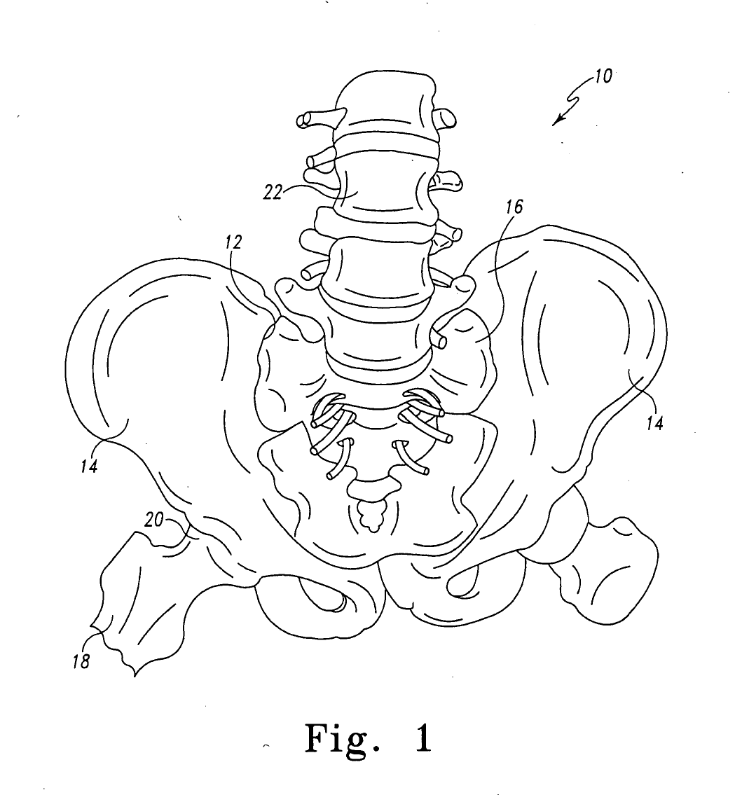

FIG. 1 illustrates in an elevation view the skeletal structure of the human pelvic region 10 having multiple joints 12 wherein the joints connect two or more adjacent bones. The pelvic region 10 consists of three bones, two iliac bones 14 and the sacrum 16. The iliac bones 14 connect to the respective femur bone 18 via a ball and socket joint 20. Thus, the iliac bones 14 translate movement from the legs. The sacrum 16 is positioned at the bottom of the spine 22 and on each side of the iliac bones 14 via sacroiliac joints 12. The sacroiliac joints 12 can be thought of as the bottom joints of the spine 22 relating to the iliac bones 14. The sacroiliac joints 12 connect the spine 22 to the pelvic region 10, and thus, the entire lower half of the skeleton.

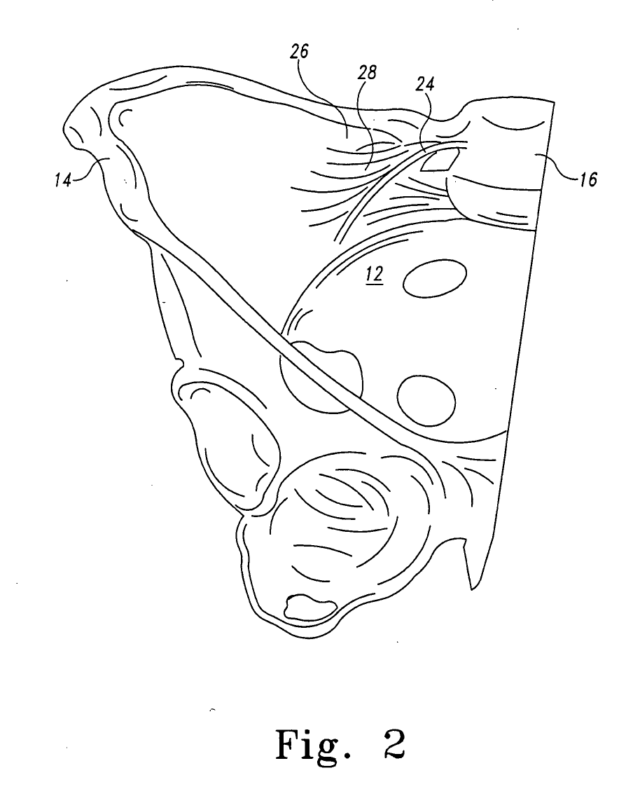

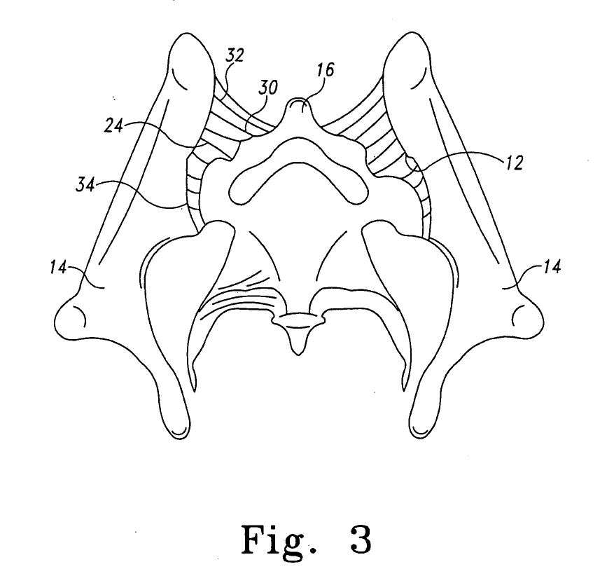

Turning to FIG. 2, ligaments 24 surround and attach to each sacroiliac joint 12 in the anterior and posterior regions such that the ligaments 24 connect the sacrum 16 to the iliac bone 14 at an inner iliac wall 26. Like all true joints, there is articular cartilage on both surface sides of each sacroiliac joint 12. Much of the integrity of each sacroiliac joint 12 depends on the ligamentous structure which can be the cause of pain and inflammation of the sacroiliac joint 12. The most commonly disrupted and/or tom ligaments 24 are the iliolumbar ligaments 28 and the posterior sacroiliac ligaments 30 (FIG. 3).

Turning to FIG. 3, each sacroiliac joint 12, illustrated in a planar cross sectional view of FIG. 2, is situated between the iliac bones 14 and the sacrum 16. As illustrated, each sacroiliac joint 12 includes a posterior region 32 and an anterior region 34. Unlike most other joints, each sacroiliac joint 12 is not designed for much motion. In fact, it is common for each sacroiliac joint 12 to become stiff and actually “lock” while the person ages over time.

Each sacroiliac joint 12 usually only moves about two to four millimeters during weight bearing and movement. This small amount of motion occurring in the sacroiliac joint 12 is described as a “gliding” type of motion. The motion is quite different than the hinge motion of the knee or the ball and socket motion of the hip. As such, each sacroiliac joint 12 is a “viscoelastic joint,” meaning that its major movement comes from giving or stretching.

One of the most common causes of problems for the sacroiliac joints 12 is an injury which strains the ligaments 24 around each sacroiliac joint 12. Straining of these ligaments 24 can lead to too much motion in the sacroiliac joint 12. The excessive motion can eventually lead to wear and tear of each sacroiliac joint 12 and pain from degenerative arthritis. Injuries such as a blow can also cause direct inflammation of the articular cartilage lining each sacroiliac joint 12 leading to degenerative arthritis in the respective sacroiliac joint 12.

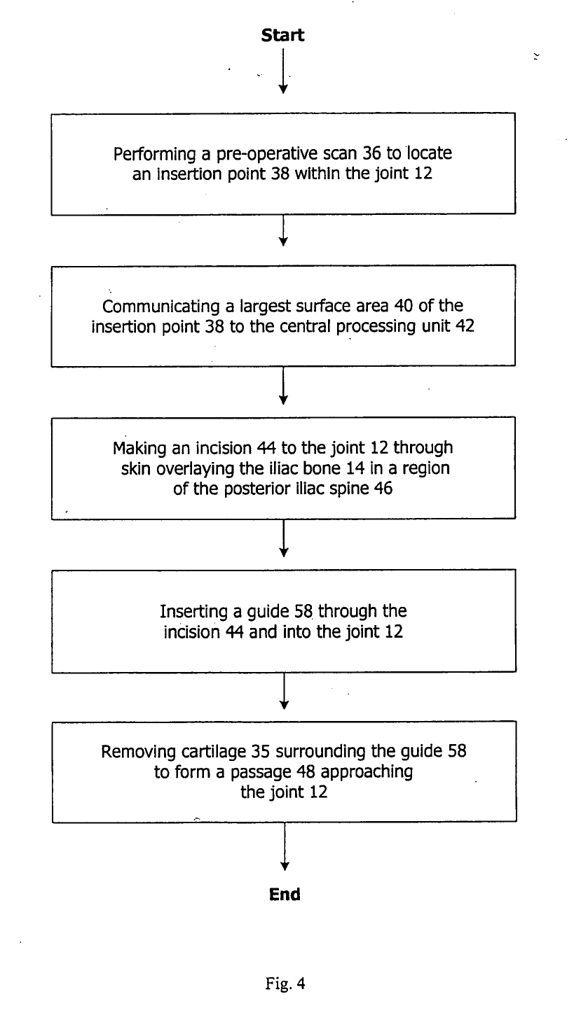

Referring to FIG. 4, a flow chart illustrates steps of approaching a particular joint 12 such as the sacroiliac joint in a minimally invasive manner. As outlined in FIG. 4, a pre-operative scan 36 scans the area of the joint 12 to locate an insertion point 38 within the joint 12 wherein the joint 12 is positioned between the first bone 14 (FIG. 1) and the second bone 16 (FIG. 1). In an embodiment, the joint 12 may be one of the sacroiliac joints while the first bone 14 may be the iliac bone and the second bone 16 may be the sacrum bone. The insertion point 38 may contain the largest surface area 40 of the joint 12 as located by the pre-operative scan 36. The pre-operative scan 36 may include a CT or MRI scan in order to locate the insertion point 38, wherein the pre-operative scan 36 communicates the insertion point 38 to a central processing unit 42.

After locating the insertion point 38 from the pre-operative scan 36, an incision 44 is made to the sacroiliac joint 12 such that the incision 44 cuts through the skin and ligaments overlying the iliac bone 14 in a region of the posterior superior iliac spine 46. As such, cutting the incision 44 to the iliac bone 14 does not invade any muscle since the region of the posterior superior iliac spine 46 is substantially free from any muscle tissue.

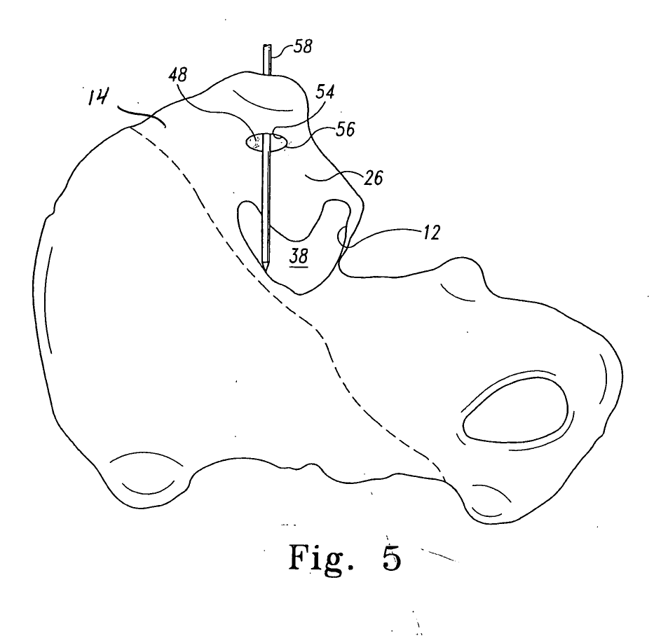

Turning to the FIG. 5 and referring to FIG. 4, a guide 58 such as a K-wire inserts through the incision 44 and into the iliac bone 14. Accordingly, the guide 58 may pass between the inner cortical layer 54 and the outer cortical layer 56 and may exit at the inner iliac wall 26. The guide 58 then proceeds to the insertion point 38 of the sacroiliac joint 12. In an embodiment, the guide 58 may communicate with central processing unit 42 in order to locate the insertion point 38.

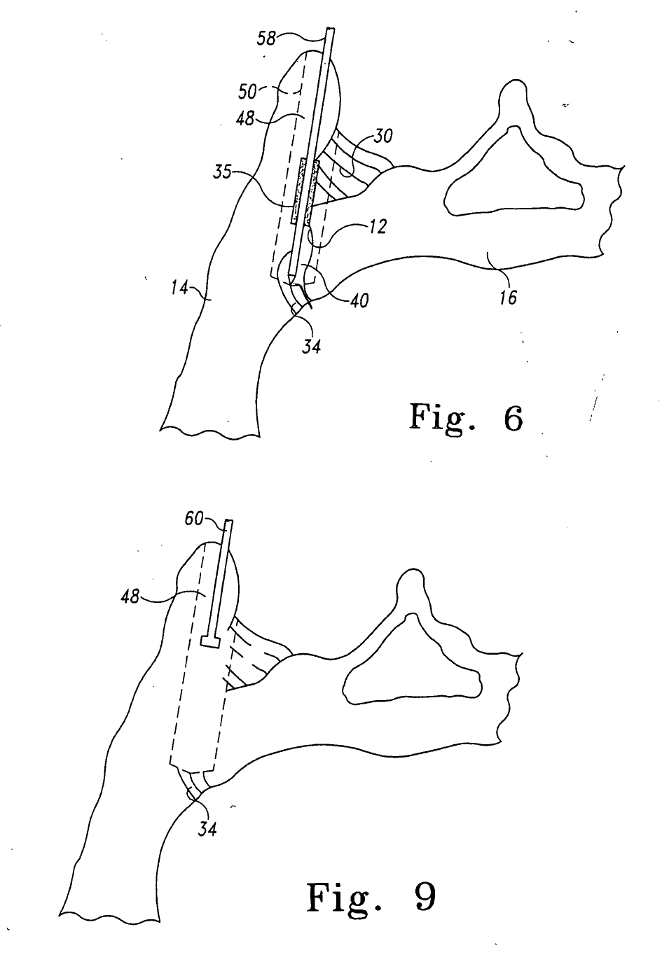

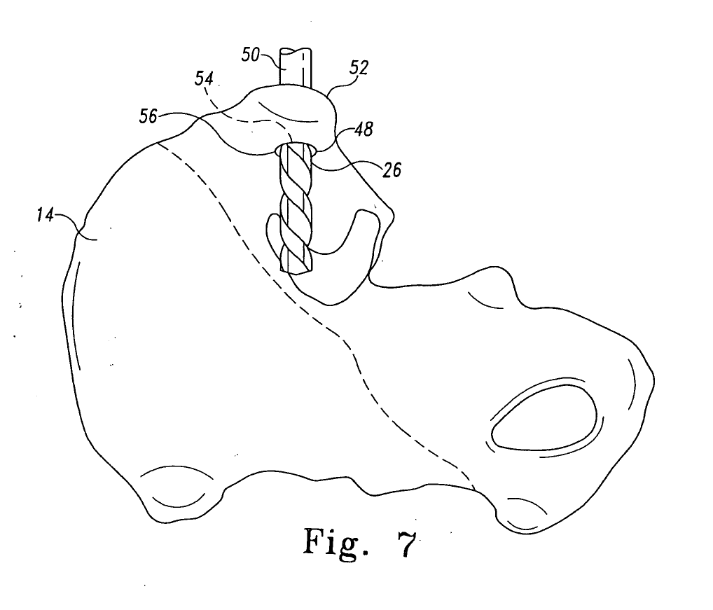

As shown in FIG. 6, the guide 58 enters the posterior sacroiliac ligaments 30 while running substantially parallel to the iliac bone 14 and the sacrum bone 16. The guide 58 is then manipulated to the proper depth and angle within the insertion point 38 of the sacroiliac joint 12 based on the pre-operative scan 36. Next, a material remover 50 such as a cannulated drill passes over the guide 58 to enlarge a passage 48 to the insertion point 38 of the sacroiliac joint 12 around the guide 58. As shown in FIG. 7, the material remover 50 may enter the iliac bone 14 via incision 44 (FIG. 4) at a posterior side of the pelvic rim 52. The remover 50 may comprise the illustrated drill while other devices such as, but not limited to, lasers may also form the passage 48. The remover 50 may pass through an inner cortical layer 54 and an outer cortical layer 56 of the iliac bone 14 to exit at the inner iliac wall 26.

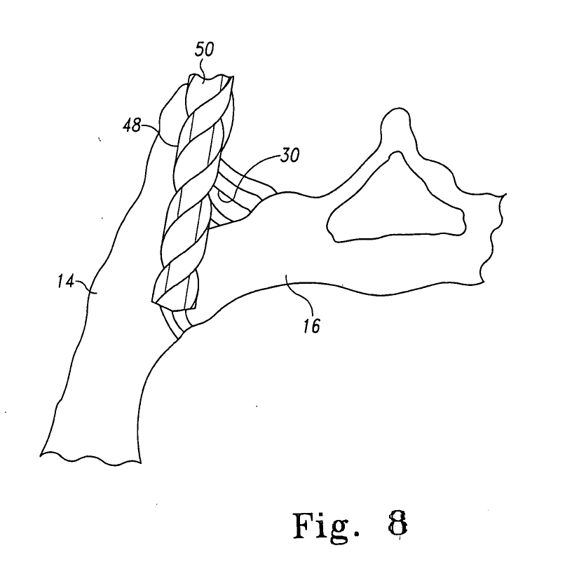

In an embodiment, material remover 50 may communicate the formation of the passage 48 with the central processing unit 42 (FIG. 4) in order to form the passage 48 to the insertion point 38 (FIG. 4). Referring to FIG. 8, the remover 50 may proceed to form the passage 48 into the posterior sacroiliac ligaments 30. As such, the passage 48 may enter the sacroiliac joint 12 while remaining substantially parallel to the iliac bone 14 and the sacrum bone 16.

Returning to FIG. 6, the enlarged passage 48 may remain free from entering the anterior region 34 of the sacroiliac joint 12. In an embodiment, though, the remover 50 may form the passage 48 into the anterior region 34. The passage 48 may be configured for placing a new or artificial joint (not shown) near the anterior region 34. Additionally, the passage 48 may be formed for removing bone pieces (not shown) having a variety of sizes. As such, the remover 50 may be sized and shaped for a variety of configurations in order to form the passage 48 accommodating the placement and/or removal of objects such as, but not limited to bone pieces, artificial joints and sponges. The cannulated remover 50 enlarges the passage 48 by removing cartilage 35 which surrounds the guide 58. The cannulated remover 50 may also remove portions of the iliac bone 14 and the sacrum bone 16 associated with the cartilage 35. In an embodiment, the material remover 50 may communicate with the central processing unit 42 in order to enlarge the passage 48 around the guide 58.

After removing the cartilage 35 and bone portions connected to the cartilage around the guide 58, the cannulated remover 50 and guide 58 are then removed to expose the passage 48 as shown in FIG. 9. A probe 60 inspects the passage 48 to determine that the passage 48 enters the insertion point 38. In an embodiment, the probe 60 may operatively communicate with the central processing unit 42 to display the clearance of the passage 48.

The present disclosure forms the passage 48 based on the pre-operative scan 36 to the insertion point 38 (FIG. 5). As configured, any fusion or non fusion device (not shown) may enter the insertion point 38 via the passage 48 in a minimally invasive manner. Once inserted into the insertion point 38, the fusion or non fusion device may engage or associate with the iliac and sacral sides of the sacroiliac joint 12.

In an embodiment, at least one other passage 48 may be formed adjacent to the initial passage 48. As such, an incision 44 is made to the sacroiliac joint 12 such that the incision 44 cuts through the skin and ligaments overlying the iliac bone 14 in a region of a posterior superior iliac spine 46. Since the region of the posterior superior iliac spine 46 is substantially free from any muscle tissue, cutting the incision 44 to the iliac bone 14 does not invade any muscle.

In this embodiment, the guide 58 inserts through the incision 44 and may pass through the inner cortical layer 54 and the outer cortical layer 56. As such, the guide 58 enters the posterior sacroiliac ligaments 30 while running substantially parallel to the iliac bone 14 and the sacrum 16. The remover 50 then passes over the guide 58 to enlarge the other passage 48 to the insertion point 38 of the sacroiliac joint 12. The remover 50 enlarges the additional passage 48 by removing cartilage 35 and associated bone portions which surround the guide 58. After removing the cartilage 35 and bone portions from around the guide 58, the remover 50 and guide 58 are then removed to expose the passage 48. A probe 60 inspects the other passage 48 to determine that the passage 48 enters the insertion point 38 while remaining adjacent to the initial passage 48.

The present disclosure, in another embodiment, approaches the joint 12 in a minimally invasive manner wherein the passage 48 is formed to the joint 12 while remaining free from violating the iliac bone 14 and/or sacrum bone 16. As such, the present disclosure relates to forming the passage 48 to the joint 12 by passing through the skin and ligaments. In this embodiment, the pre-operative scan 36 scans the area of the joint 12 to locate the insertion point 38 within the joint 12 wherein the joint 12 is positioned between the first bone 14 and the second bone 16.

After locating the insertion point 38 from the pre-operative scan 36, the incision 44 is made to the joint 12 such that the incision 44 cuts through the skin and ligaments overlying the joint 12 without contacting any bone. The guide 58 then enters the incision 44 while passing through the ligaments to directly approach the joint 12 while remaining free from entering any bone. The guide 58 is then manipulated to the proper depth and angle within the insertion point 38 based on the pre-operative scan 36. Next the remover 50, such as the cannulated drill, passes over the guide 58 to form the passage 48 to the insertion point 38 of the joint 12. In an embodiment, the remover 50 may form the passage 48 without contacting any bone when the guide 58 directly enters the sacroiliac joint 12 from the incision 44. As such, the passage 48 enters the joint 12 in a minimally invasive approach with respect to the iliac bone 14 and the sacrum bone 16 when the guide 58 directly enters the sacroiliac joint from the incision 44.

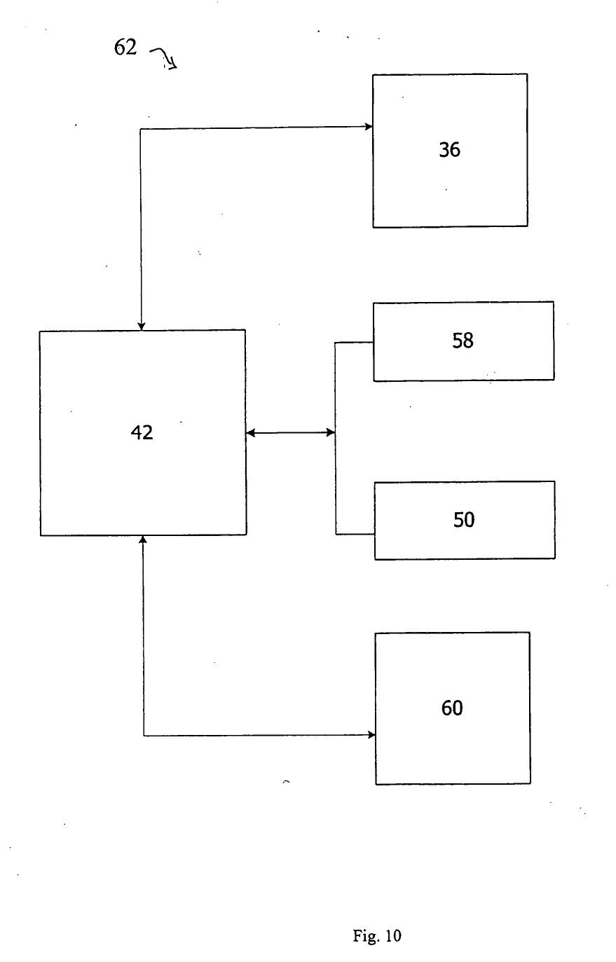

Turning to FIG. 10 and referring to FIGS. 1-9, a system 62 for approaching the sacroiliac joint 12 is illustrated in schematic form. The system 62 comprises the central processing unit 42, the pre-operative scanner 36, the guide 58, the material remover 50 and the probe 60.

In an embodiment, the scanner 36 is adapted to analyze the sacroiliac joint 12 for the insertion point 38 within the sacroiliac joint 12 while operatively communicating with the central processing unit 42, wherein the insertion point 38 may be positioned within the posterior region 32 of the sacroiliac joint 12. The scanner 36 may include a CT or MRI device in order to analyze the sacroiliac joint 12. Once determined, the pre-operative scanner 36 communicates the insertion point 38 to the central processing unit 42.

The guide 58 is adapted to insert through the incision 44 and into the sacroiliac joint 12. Thus, the guide 58 inserts substantially parallel to the iliac bone 14 and the sacrum bone 16. The material remover 50, meanwhile, is adapted to form the passage 48 around the guide 58 through the iliac bone 14 and into the post superior ligaments 30 of the sacroiliac joint 12. The remover 50 may communicate the formation of the passage 48 to the central processing unit 42 to confirm the passage 48 being formed to the sacroiliac joint 12. The remover 50 is adapted to enlarge the passage 48 around the guide 58 by removing the cartilage 35 and the bone portions surrounding the guide 58. The guide 58 may operatively communicate its position within the passage 48 to the central processing unit 42.

After removing the guide 58, the probe 60, adapted to may be inserted within the passage 48, analyzes the passage 48 to determine that the insertion point 38 has been entered. The probe 60 may operatively communicate to the central processing unit 42 that, in an embodiment, the anterior region 34 of the sacroiliac joint 12 remains free from contact or that the anterior region 34 has been entered. As such, the system 62 is configured to form the passage 48 in a minimally invasive approach to the sacroiliac joint 12.

In an embodiment, the present disclosure relates to the system 62 approaching the joint 12 in a minimally invasive manner wherein the passage 48 is formed with the joint without violating or minimally violating any bones. As such, the system 62 relates to forming the passage 48 to the joint 12 by passing through the skin and ligaments. In this embodiment, the scanner 36 analyzes the joint 12 for the insertion point 38. Once determined, the pre-operative scanner 36 communicates the insertion point 38 to the central processing unit 42. The incision 44 is made to the joint 12 such that the incision 44 cuts through the skin and ligaments overlying the bone. The guide 58 then enters the incision 44 while passing through the ligaments to approach the joint 12 while remaining free from entering any bone. The guide 58 may operatively communicate its position to the central processing unit 42. Next, the remover 50, in communication with the central processing unit 42 passes over the guide 58 to form the passage 48 to the insertion point of the joint 12. Thus, the passage 48 enters the joint 12 based on the pre-operative scan 36 in a minimally invasive approach free from contacting any bone.

While the present disclosure describes the sacroiliac joint, the iliac bone and the sacrum bone, it is understood that the present disclosure is not limited to any particular joint in the body. While the concepts of the present disclosure have been illustrated and described in detail in the drawings and foregoing description, such an illustration and description is to be considered as exemplary and not restrictive in character, it being understood that only the illustrative embodiments have been shown and described and that all changes and modifications that come within the spirit of the disclosure are desired to be protected by the following claims.

I claim:

1. A method of approaching a joint which connects a first bone and a second bone, comprising:
cutting an incision to the joint;
inserting a guide through the incision and into the joint; and
forming a passage around the guide wherein the passage approaches the joint while remaining substantially parallel to the first bone and the second bone.

2. The method of approaching a joint according to claim 1, wherein the joint is a sacroiliac joint.

3. The method of approaching a joint according to claim 2, wherein the first bone is an iliac bone.

4. The method of approaching a joint according to claim 3, wherein the second bone is a sacrum bone.

5. The method of approaching a joint according to claim 3, wherein cutting the incision comprises cutting through skin overlaying the iliac bone in a region of a posterior superior iliac spine.

6. The method of approaching a joint according to claim 5, wherein inserting the guide comprises passing the guide through the posterior superior iliac spine and into the joint.

7. The method of approaching a joint according to claim 1, further comprising operating a pre-operative scan prior to cutting the incision to locate an insertion point within the joint.

8. The method of approaching a joint according to claim 7, wherein the insertion point contains a largest surface area of the joint.

9. The method of approaching a joint according to claim 4, wherein inserting the guide comprises passing the guide through the iliac bone by entering the iliac bone at an iliac rim.

10. The method of approaching a joint according to claim 9, wherein inserting the guide comprises passing the guide between an inner cortical layer and an outer cortical layer of the iliac bone.

11. The method of approaching a joint according to claim 10, further comprising exiting the guide at an inner iliac wall.

12. The method of approaching a joint according to claim 11, further comprising passing the guide into posterior sacroiliac ligaments.

13. The method of approaching a joint according to claim 12, further comprising passing the guide into the sacroiliac joint while remaining parallel to the iliac bone and the sacrum bone.

14. The method of approaching a joint according to claim 1, further comprising removing the guide after forming the passage.

15. A method of approaching a sacroiliac joint which connects the iliac bone and the sacrum bone, comprising:
(a) cutting an incision through skin in a region of the posterior superior iliac spine overlaying the iliac bone;
(b) inserting a guide through the iliac bone and into the sacroiliac joint;
(c) removing cartilage and bone portions surrounding the guide to form a passage;
(d) removing the guide after forming the passage; and
(e) repeating steps a-d for forming additional passages into the sacroiliac joint wherein the passages approach a largest surface area of the sacroiliac joint.

16. The method of approaching a sacroiliac joint according to claim 15, wherein cutting the incision comprises cutting to the iliac bone free from invading muscle.

17. The method of approaching a sacroiliac joint according to claim 15, wherein removing the cartilage and bone portions comprises drilling around the guide through the iliac bone by entering the iliac bone at an iliac rim.

18. The method of approaching a sacroiliac joint according to claim 15, wherein inserting the guide comprises passing the guide between an inner cortical layer and an outer cortical layer of the iliac bone.

19. The method of approaching a sacroiliac joint according to claim 15, further comprising inspecting the largest surface area of the sacroiliac joint after removing the guide.

20. A system for approaching a sacroiliac joint which connects an iliac bone and a sacrum bone, comprising:
a central processing unit;
a scanner in communication with the central processing unit, the scanner being adapted to analyze the sacroiliac joint for an insertion point within the sacroiliac joint;
a guide in communication with the central processing unit, the guide being adapted to insert into the insertion point; and
a remover adapted to form a passage around the guide and into the insertion point wherein the passage is configured to allow access to the insertion point.

21. The system for approaching a sacroiliac joint according to claim 20, further comprising a probe in communication with the central processing unit wherein the probe is adapted to analyze the passage.

22. The system for approaching a sacroiliac joint according to claim 20, wherein the insertion point is positioned in a posterior region of the sacroiliac joint.

23. The system for approaching a sacroiliac joint according to claim 20, wherein the remover comprises a cannulated drill.

24. The system for approaching the sacroiliac joint according to claim 20, wherein the guide is adapted to be inserted into the insertion point while remaining free from contacting bone.
